(19) United States
(12) Patent Application Publication
(10) Pub. No.: US 2002/0082708 A1
(43) Pub. Date: Jun. 27, 2002

(54) Penile pump with side release mechanism

(75) Inventors: John G. Almli, Chaska, MN (US); Charles C. Kuyava, Eden Prairie, MN (US); Randy Morningstar, Brooklyn Park, MN (US)

Correspondence Address: Jeffrey J Hohenshell, American Medical Systems Inc, 10700 Bren Road West, Minnetonka, MN, 55343, US

(73) Assignee: American Medical Systems Inc.

(21) Appl. No.: 09/749,075

(22) Filed: Dec. 27, 2000

(51) Int. Cl. A61F 2/04; A61F 2/26

(52) U.S. Cl. 623/23.67; 623/23.68

(57) ABSTRACT

A pump and valve assembly for an implantable prothsesis is provided with an internal actuating bar positioned such that when any portion of the housing is compressed, the check valves within are opened allowing for deflation of the cylinders. The pump and valve assembly also includes a textured surface over a portion of the housing to allow for quick identification of the component, as well as to make it easier for the patient to grasp it. The pump bulb has a different size than than the valve assembly, further facilating the differention between the two based on tactile perception.

### BACKGROUND OF THE INVENTION

The present invention relates to a pump and valve assembly for an implantable prosthesis. More specifically, the present invention relates to a pump and valve assembly configured to facilitate the manipulation and actuation of an implantable prosthesis.

One common treatment for male erectile dysfunction is the implantation of a penile prosthesis. Such a prosthesis typically includes a pair of inflatable cylinders, which are fluidly connected to a reservoir (typically liquid filled) via a pump and valve assembly. The two cylinders are normally implanted into the corpus cavernosae of the patient and the reservoir is typically implanted into the patient's abdomen. The pump assembly is implanted in the scrotum.

During use, the patient actuates the pump and fluid is transferred from the reservoir through the pump and into the cylinders. This results in the inflation of the cylinders and thereby produces the desired penis rigidity for a normal erection. Then, when the patient desires to deflate the cylinders, a valve assembly within the pump is actuated in a manner such that the fluid in the cylinders is released back into the reservoir. This deflation then returns the penis to a flaccid state.

Presently, the pump and valve assembly used in such implantable prostheses share certain similar characteristics. For example, they include fluid pathways allowing the flow of fluid to and from the reservoir, as well as to and from the cylinders. This fluid flow is controlled by one or more check valves positioned in the fluid pathways within the housing of the assembly.

A compressible pump bulb is also attached to the housing and is in fluid communication with the various fluid pathways therethrough. In order to inflate the cylinders, the compressible pump bulb is actuated by the patient thereby urging fluid past the check valves into the cylinders. In order to deflate the cylinders, the valve housing is grasped and squeezed (through the patient's tissue), causing the various check valves to unseat and allow fluid to flow back to the reservoir.

Since the pump and valve assembly is positioned within the patient's scrotum, the various components of the assembly must be small. As a result, manipulation of the pump and valve assembly is sometimes difficult. For example, patients requiring the use of penile prosthesis discussed herein are oftentimes elderly and have a reduced dexterity as a result of aging. Thus, in some instances, even locating the device within the tissue can be a challenge, let alone identifying the correct portion of the assembly to actuate. More specifically, with some patients, it may be difficult to determine whether the housing portion of the assembly that leads to release or deflation of the cylinders is being grasped or whether the bulb portion, which would be used to inflate the cylinders, is being grasped.

In this connection, it should be noted that the length of the valve assembly is determined (at least in one direction) by the size of the various check valves and the distance such valves must move in order to open and close the various fluid passageways. As a result, such a pump and valve assembly typically is longer in a direction parallel with the check valves. Moreover, in order to release the check valves in an assembly configured in this manner, the patient must grasp the narrower, shorter sidewalls of the assembly and compresses them together. Since such a configuration can present challenges insofar as the spring tension of the check valves at the time of desired deflation is typically at a maximum while the surface area of the assembly which must be compressed in order to cause such deflation is at a minimum. This condition can lead to a situation where the patient has difficulty actually compressing the assembly, or in extreme circumstances, actually loses grip of the assembly during such attempts at deflation.

Thus, although existing devices function with extreme efficiency and reliability, for some patients it appears there is a desire for a pump and valve assembly in an implantable prosthesis that improves operative manipulation of the assembly.

### BRIEF SUMMARY OF THE INVENTION

The present invention provides various features which taken alone or in combination with one another provide for an improved pump and valve assembly for an implantable prosthesis. The present pump and valve assembly includes a pump bulb that must be differentiated from the valve housing when inflation of the cylinders is desired. The pump bulb itself has dimensions that are somewhat different than the remainder of the housing. However, to supplement differentiation between the bulb and the valve housing, the valve housing is provided with a textured surface so that even through tissue the patient is able to readily discern which area comprises the pump bulb and which area comprises the valve housing. This is important in that the pump bulb is compressed for inflation while the valve housing is compressed for deflation.

The pump assembly of the present invention is also configured such that it has a length longer than its width, with its internal check valves running parallel with the length. To release fluid from the inflated cylinders, the internal check valves are actuated so that they move in a direction parallel to the length, until they open. To achieve this action directly, the opposing sides of the width of the valve housing are compressed. This compression causes actuation of the internal check valves.

In addition, an actuating bar is positioned within the valve housing parallel with and extending along at least one of the sides of the length. An arm attached to the actuating bar extends along a portion of one of the sides of the width in close proximity to the tip of one of the check valves. Thus, the configuration of the actuating bar causes it to engage and open the check valve allowing fluid to flow from the cylinder to the reservoir. Furthermore, the patient can grasp the valve housing in virtually any orientation and when pressure is applied, the actuating bar will act either directly or indirectly to open the appropriate check valves. Thus, so long as the patient grasps any portion of the pump and valve assembly other than the pump bulb, compression will result in the desired opening of the check valves, which will allow the cylinders to deflate.

Furthermore, since the patient can grasp the valve housing along the sides of the length, i.e., surfaces with larger surface area, less pressure need be applied to achieve the successful opening of the check valves. In other words, by increasing the surface area that is engaged by the patient's fingers and appropriately positioning the actuating bar, less force need be exerted by the patient to achieve the desired result.

The textured surface of the valve housing not only helps the patient identify the correct portion of the pump and valve assembly to actuate, it also serves to prevent slippage once the patient begins to compress the housing. Thus, what is achieved is an efficient and ergonomic pump and valve assembly for an implantable prosthesis. The pump and valve assembly can advantageously be formed from a minimal number of components. That is, all that need be molded are a valve block and a corresponding pump bulb that surrounds the valve block. The various check valves can be inserted into the valve block and then placed within the interior of the pump bulb, thus forming a completed assembly. This results in certain manufacturing efficiencies, thus reducing both cost and time of production.

### BRIEF DESCRIPTION OF THE DRAWINGS

FIG. 1 is a perspective view of a pump and valve assembly according to the present invention.

FIG. 2 is a front sectional view of the pump and valve assembly illustrated in FIG. 1.

FIG. 3 is a top sectional view of the pump and valve assembly illustrated in FIG. 1, shown in a state where the cylinders are being deflated.

FIG. 4 is a top sectional view of the pump and valve assembly illustrated in FIG. 1, shown in a state where the check valves are in a deactivated position.

FIG. 5 is a top sectional view of the pump and valve assembly illustrated in FIG. 1, shown in a state where the check valves are in a pumping position.

FIG. 6 is a side sectional view of the pump and valve assembly illustrated in FIG. 1.

### DETAILED DESCRIPTION OF THE INVENTION

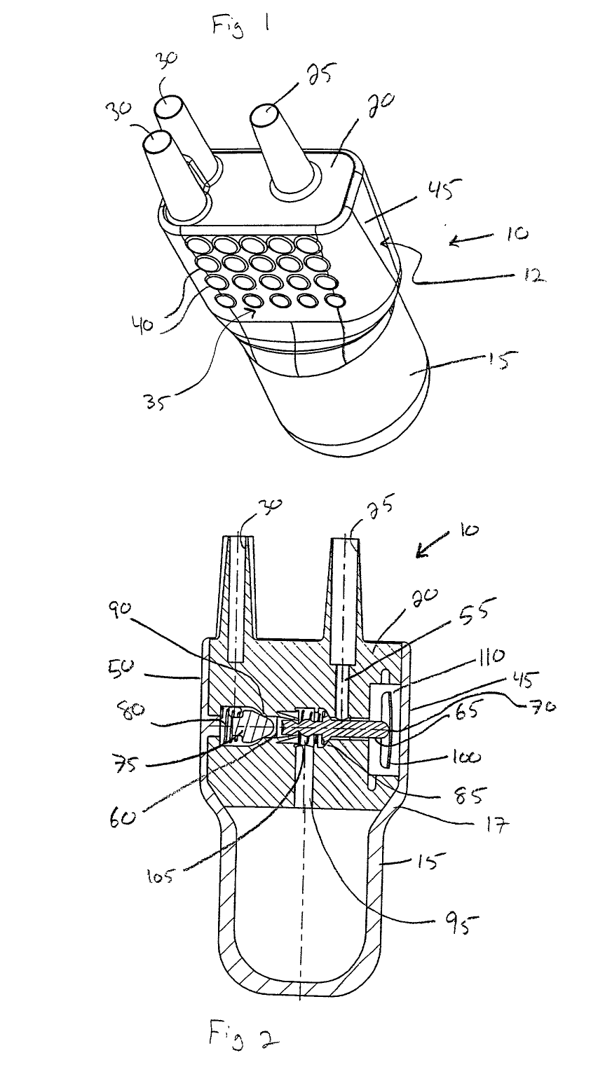

Referring to FIG. 1, a pump and valve assembly is illustrated and generally referred to as 10. Pump and valve assembly 10 includes two different sections: valve housing 12 and pump bulb 15. Pump bulb 15 is a compressible member, defining a chamber more clearly shown in FIG. 2. Valve housing 12 is fluidly coupled to pump bulb 15 and contains the various other working components of pump and valve assembly 10. Pump and valve assembly 10 will be fluidly coupled to a reservoir and a pair of cylinders (not shown). This is accomplished through tubing connected to reservoir coupling 25 and cylinder couplings 30, which are integral with valve housing 12. Pump and valve assembly 10 is configured such that pump bulb 15 extends from one end of valve housing 12, while reservoir coupling 25 and cylinder couplings 30 extend from the other. Thus, when implanted in the patient, reservoir coupling 25 and cylinder couplings 30 and the fluid tubing they are coupled to are oriented toward the patient's abdomen, while the pump bulb 15 is disposed in the opposite direction. Therefore, when pump bulb 15 is grasped by a patient, there is no interference from or contact with the tubing coupled to reservoir coupling 25 and cylinder couplings 30.

Valve housing 12 is illustrated as being generally rectangular, having a first major panel 35 that is longer than first minor panel 45. The length of first major panel 35 is determined by the distance required to incorporate the various check valves described below and allow their proper functioning. Likewise, first minor panel 45 need only be long enough to incorporate the width of these check valves and once again allow their proper functioning. Of course, some consideration can be given to the optimal diameter of the fluid tubing and couplings connecting pump and valve assembly 10 to the reservoir and cylinders. Though shown as being generally rectangular, valve housing 12 can take on any configuration (and dimension) so long as the check valves contained therein operate correctly. The illustrated configuration generally minimizes the volume required for valve housing 12 to operate effectively. Thus, the net result is that first major panel 35 is generally longer than first minor panel 45.

Referring to FIGS. 1 and 2, the internal configuration of pump and valve assembly 10 will be described. Two separate molded components are utilized to form pump and valve assembly 10. That is, valve block 20 is combined with shell 17 to form the completed unit. Pump bulb 15 and valve housing 12 are a single, integral unit referred to as shell 17 that substantially surrounds valve block 20. As illustrated, shell 17 includes valve housing 12, which surrounds valve block 20. Alternatively, shell 17 could be a smaller component that does not surround valve block 20, but is simply coupled to it. In either case, only two molded components need be provided to complete the device. These components can be formed from silicone or any other appropriate material.

The use of only two molded components to form pump and valve assembly 10 is advantageous. Previous devices generally have four or more molded components which must be individually put together. Only two components can be bonded in a single step. Bonding includes heating, using adhesive, or various other joining techniques. The two bonded components then take time to set up before the next component can be added. Thus, a four component device results in a fairly long manufacturing process having increased costs associated therewith.

With the present device, valve block 20 is molded and the various valve components are inserted into place. Shell 17 is then attached and bonded. Thus, only a single bonding or adhering step is required to complete the product. This greatly increases throughput, decreases costs, and decreases manufacturing time without sacrificing quality or durability.

Located within valve block 20 are a plurality of fluid passageways coupling reservoir coupling 25 and cylinder couplings 30 to pump bulb 15 through bulb passageway 95 via medial passageway 60. Disposed within medial passageway 60 are two springactuated poppets: a reservoir poppet 65 and a cylinder poppet 75, which respectively and selectively abut reservoir poppet valve seat 85 and cylinder poppet valve seat 90. Cylinder poppet 75 is a relatively simple conical-shaped check valve. Reservoir poppet 65 is an elongated member having a somewhat more complicated shape. The configuration of reservoir poppet 65, along with the configuration of valve block 20 along medial passageway 60 is designed to allow the proper operation of the poppets while also preventing spontaneous inflation. The functionality and operability of this arrangement is discussed in co-pending application, filed concurrently herewith, entitled Pressure Based Spontaneous Inflation Inhibitor In A Pump For An Inflatable Prosthesis, the entire disclosure of which is herein incorporated by reference.

During a compression of pump bulb 15, fluid is forced from the internal chamber of pump bulb 15 through bulb passageway 95, causing cylinder poppet 75 to open and allow fluid to flow through cylinder couplings 30 into the respective cylinders. When pump bulb 15 is released, cylinder poppet 75 closes under spring pressure. The vacuum generated by pump bulb 15 causes reservoir poppet 65 to unseat itself and allow fluid to flow from the reservoir through reservoir coupling 25 so that fluid once again fills pump bulb 15. Repeated compressions are performed to entirely inflate the cylinders to the patient's satisfaction.

When it is desired to deflate the cylinders, the patient compresses valve housing 12 by squeezing first minor panel 45 towards second minor panel 50. As this occurs, the outer wall of valve housing 12 engages actuating bar arm 130 which engages reservoir poppet tip 70, causing reservoir poppet 65 to unseat itself as well as unseating cylinder poppet 75. Fluid is then able to flow from the cylinders to the reservoir through medial passageway 60. When satisfactorily deflated, the patient releases valve housing 12, allowing reservoir poppet 65 and cylinder poppet 75 to reseat themselves and prevent fluid flow.

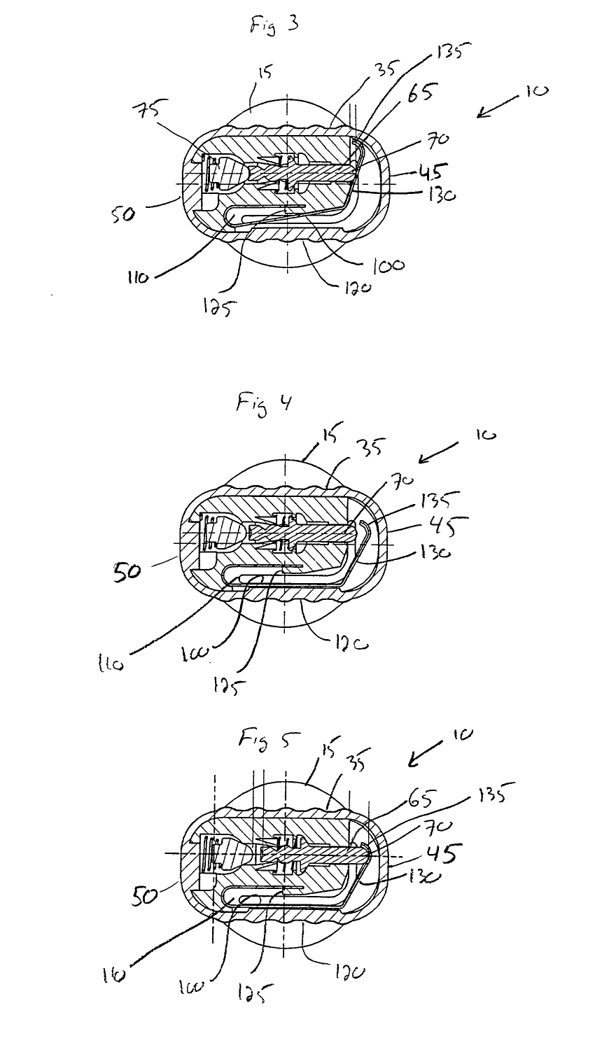

To perform the above described deflation process, the patient may compress first minor panel 45 and second minor panel 50. In some patients, however, it may be difficult to achieve this compression because of the relatively small size of first and second minor panels 45 and 50. Likewise, it may be difficult for certain patients to grasp valve housing 12 in this manner since valve housing 12 may slip out of position between the patient's fingers. Thus, the present pump and valve assembly 10 provides an actuating bar 100 that allows the patient to grasp the first major panel 35 and second major panel 120 (as illustrated in FIGS. 3-5).

Referring to FIG. 3, the operation of actuating bar 100 is illustrated. Actuating bar 100 is disposed within valve block 20 by frictionally securing one end of actuating bar 100 into valve block interface 125 which securely holds it in place. Actuating bar 100 extends substantially along the length of major panel 120. Actuating bar arm 130 is integrally coupled with actuating bar 100 and generally extends substantially along the length of first minor panel 45. Actuating bar 100 is comprised of a suitable material, such as stainless steel or plastic. FIG. 3 illustrates a configuration of actuating bar 100 when a patient is compressing valve housing 12. The configuration illustrated in FIG. 4 is that of a deactivated state. In this state, the patient does not intend to inflate (nor deflate) the cylinders. The relationship between reservoir poppet 65 and valve block 20 in the area of medial passageway 60 is such that spontaneous inflation is prevented. FIG. 5 illustrates a pumping state. Reservoir poppet 65 is moved to the right (as illustrated) and tip 70 abuts arm 130. When pump bulb pressure is sufficient, cylinder poppet 75 will be unseated. FIGS. 4 illustrates the position of actuating bar 100 in a deactivated state, that is, when the patient is not compressing valve housing 12.

Returning to FIG. 1, major panels 35 and 120 contain a textured surface 40, containing a plurality of raised sections. These raised sections make it easy for the patient to identify and distinguish valve housing 12 from pump bulb 15 and also allow the patient to grasp it better. Furthermore, because major panels 35 and 120 are relatively large in comparison to minor panels 45 and 50, it is easier for the patient to grasp and compress these major panels 35 and 120.

Referring once again to FIG. 3, when major panels 35 and 120 are compressed towards one another, actuating bar 100 is deflected from the position illustrated in FIG. 4 to the position illustrated in FIG. 3. Thus, by engaging reservoir poppet tip 70, actuating bar arm 130 forces reservoir poppet 65 to move towards and open cylinder poppet 75. More specifically, actuating bar 100 is generally parallel with second major panel 120 in the deactivated stage. When engaged, actuating bar 100 is deflected towards first major panel 35. Because of the angle between actuating bar 100 and actuating bar arm 130, actuating bar arm 130 is caused to move towards reservoir poppet tip 70, as well as first major panel 35. Insofar as this movement is defined by the internal wall of valve housing 12, actuating bar arm 130 moves to the position illustrated in FIG. 3, engaging and opening reservoir poppet 65. Of course, this does not preclude the patient from grasping first minor panel 45 and second minor panel 50 and compressing them towards one another. If this is done, reservoir poppet 65 will likewise be effectively unseated. As such, it should be noted that the patient can grasp valve housing 12 in numerous orientations and a compression will effectively either directly engage reservoir poppet 65 or cause actuating bar 100, and more particularly actuating bar arm 130 to engage and open reservoir poppet 65. Thus, the patient need not maintain any particular orientation of valve housing 12 while deflating the cylinders. That is, any grip achieved on the valve housing 12 can be utilized to effectively open the poppets.

The configuration of major panels 35 and 120, including textured surface 40, will allow patients to easily identify the portion of valve housing 12 having a larger surface area and to grip it more effectively. When doing so, it may seem to the patient that less force need be applied in order to unseat reservoir poppet 65. That is, the spring tensions involved are constant for cylinder poppet 75 and reservoir poppet 65. However, because of the larger surface area of major panels 35 and 120, as compared to minor panels 45 and 50, the patient need apply less force in order to successfully actuate the device.

The configurations illustrated in FIGS. 4 and 5 differ only in that reservoir poppet 65 is in different positions with respect to valve block 20, depending upon whether the device is in a deactivated state as in FIG. 4 or in a pumping state as in FIG. 5. This is more a characteristic of the spontaneous inflation preventing mechanism as mentioned above, rather than being directly related to the operation of actuating bar 100. Of note, actuating bar arm 130 is configured to receive reservoir poppet tip 70 during the pumping stage as illustrated in FIG. 5. That is, during the compression of pump bulb 15 fluid pressure will force reservoir poppet 65 to its right most position as illustrated in FIG. 5. Because of the configuration of actuating bar arm 130 in its unbiased position, it will not interfere with this operation.

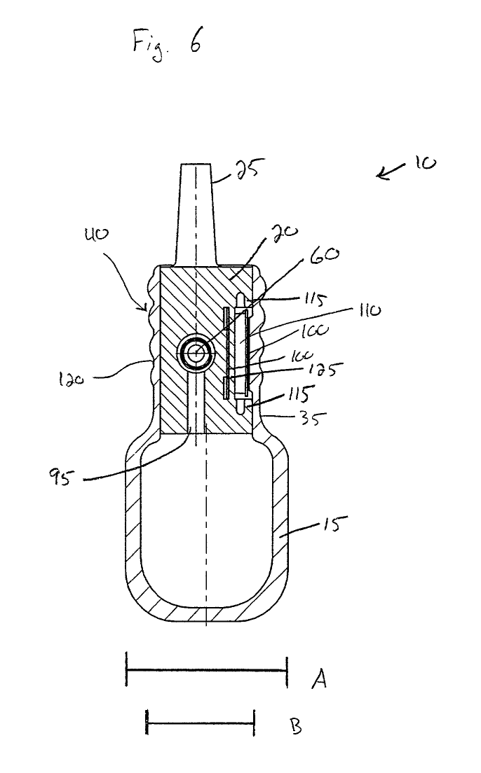

FIG. 6 illustrates a side sectional view of pump and valve assembly 10. Actuating bar 100 only extends along a portion of valve block 20. When a patient engages first major panel 120, actuating bar 100 will be relatively small in comparison to the surface area defined by the patient's finger. To further facilitate the ease with which the patient can compress actuating bar 100 and effectively unseat reservoir poppet 65, valve block 20 is enhanced by valve block tabs 115, which help define valve block recess 110 within which actuating bar 100 is seated. Thus, when the patient engages first major panel 35, moving it towards second major panel 120, this movement is enhanced by the flexibility of valve block tabs 115 allowing a larger portion of first major panel 35 to deflect into valve block recess 110.

The ease with which the patient can identify, grasp and compress the relevant portion of pump and valve assembly 10, may ultimately determine the patient's overall satisfaction with the device. FIG. 6 illustrates yet another factor that serves to facilitate this. The width of pump bulb 15 is defined as A, while the width of valve housing 12 is defined as B. Notably, the width A of valve housing 12 is smaller than the width A of pump bulb 15. The relevant factor is that pump bulb 15 is sized differently than valve housing 12. It does not matter which component is larger or smaller.

Thus, when the patient grasps pump and valve assembly 10, there are several factors that can be utilized to determine which portion the patient is grasping. First, the orientation of pump bulb 15 towards the bottom is an initial indicator. The textured surface 40 of the major panels 35 and 120 is a secondary indicator and the relative size difference between pump bulb 15 and valve housing 12 is a tertiary indicator. These components also work together along with actuating bar 100 to make it easier for the patient to compress valve housing 12 and open the internal poppets, allowing the cylinders to be deflated. This is accomplished because major panels 35 and 120 are larger and easier to grasp and their compression towards one another actuates actuating bar 100 which in turn actuates and opens reservoir poppet 65. The textured surface 40 makes it easier for the patient to grip valve housing 12 during this process. Finally, the configuration of actuating bar 100 can be configured to provide positive feedback to the patient that they are successfully opening the valves to allow for deflation. That is, actuating bar 100 can be provided with a bent area configured such that when actuating bar 100 is actuated, it will cause a clicking sensation that is audibly or physically sensed by the patient to let them know that they have sufficiently compressed valve housing 12. Other identifying devices or configurations could be used as well.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited in the particular embodiments that have been described in detail therein. Rather, reference should be made to the appended claims as indicative of the scope and content of the present invention.

What is claimed is:

1. A pump assembly for an implantable prosthesis, comprising:
a housing having an outer wall with at least a portion of the outer wall being compressible;
a first flow valve positioned within the housing and having a seated and an unseated position; and
a bar positioned within the housing and moveable between a first and a second position so that when the bar is moved from the first position to the second position the bar causes the first flow valve to move from the seated to the unseated position.

2. The pump assembly of claim 1, wherein the outer wall further comprises:
a first compressible side wall positioned to intersect an axis defined by a path of travel of the first flow valve from the seated to the unseated position;
a second compressible side wall adjacent to the first compressible sidewall, located such that a first portion of the bar is adjacent to the first compressible side wall and a second portion of the bar is adjacent to the second compressible side wall so that if either the first or the second compressible sidewall is compressed, the bar is caused to engage the first flow valve and move the first flow valve from the seated to the unseated position.

3. The pump assembly of claim 2 wherein the housing has a substantially rectangular configuration with the first compressible side wall being shorter than the second compressible sidewall.

4. The pump assembly of claim 3 wherein the second portion of the bar is substantially parallel with the second compressible side wall when the second compressible side wall is in an uncompressed state.

5. The pump assembly of claim 4 wherein an interior angle formed between the first portion of the bar and the second portion of the bar is obtuse.

6. The pump assembly of claim 2 wherein the bar is formed from stainless steel.

7. The pump assembly of claim 2 wherein the bar is formed from plastic.

8. The pump assembly of claim 3 wherein the first portion of the bar includes a curved free end wherein a curvature of the free end matches a curvature of the first flow valve.

9. The pump assembly of claim 8 wherein the curvature of the free end also matches a curvature of an interior portion of the outer wall.

10. The pump assembly of claim 2, further comprising:
a pump bulb coupled to the housing, wherein the pump bulb has a first exterior texture and the housing has a second exterior texture that is different than the first exterior texture.

11. The pump bulb of claim 10 wherein the second exterior texture includes a plurality of raised panels.

12. The pump bulb of claim 11 wherein the raised panels are circular.

13. The pump assembly of claim 2 further comprising a second flow valve positioned such that when the first flow valve is moved from the seated to the unseated position, the first flow valve contacts the second flow valve and moves the second flow valve from a seated to an unseated position.

14. An implantable prosthesis, comprising:
a housing having a generally rectangular configuration defined by a first and a second minor sidewall and a first and a second major sidewall wherein the major sidewalls are longer than the minor sidewalls, wherein at least one of the major sidewalls and at least one of the minor sidewalls is compressible;
a first flow valve located within the housing and oriented to be generally parallel with the major sidewall and perpendicular to the minor sidewalls; and
a bar located within the housing having a first portion that is substantially parallel to the compressible major sidewall and a second portion that is angled toward the compressible minor sidewall in proximity to the first flow valve so that a compression of either the compressible major sidewall or the compressible minor sidewall causes the bar to move so that the second portion contacts the first flow valve and moves it from a seated position to an unseated position.

15. The implantable prosthesis of claim 14, further comprising:
a valve block located within the housing that supports and retains the first flow valve and retains the first portion of the bar;
a recess within the valve block to receive the first portion of the bar as it is moved by a compression of either the compressible major or minor sidewall; and
a tab formed by a portion of the valve block wherein the tab is deflectable into the recess.

16. The implantable prosthesis of claim 14, further comprising:
a pump bulb coupled to the housing, wherein the pump bulb has a first exterior texture and the housing has a second exterior texture that is different than the first exterior texture.

17. The implantable prosthesis of claim 16 wherein the second exterior texture includes a plurality of raised panels.

18. The implantable prosthesis of claim 17 wherein the raised panels are circular.

19. The implantable prosthesis of claim 14 wherein the bar is formed from stainless steel.

20. A method of using an inflatable implanted prosthesis comprising:
implanting an inflatable prosthesis into a patient, said prosthesis including a pump assembly;
inflating said prosthesis with a pump included in said pump assembly; and,
randomly selecting any opposing surfaces on the periphery of said pump assembly;
physically compressing said randomly selected opposing surfaces of said pump assembly so as to deflate said prosthesis.

21. A method as set forth in claim 20, wherein compressing includes moving a check valve internal to said pump into a position to allow said prosthesis to become deflated.

22. A method as set forth in claim 21, wherein compressing includes directly contacting said check valve through patient tissue.

23. A method as set forth in claims 22, wherein said compressing includes indirectly contacting said check valve outside of patient tissue.

24. A method as set forth in claim 22, wherein compressing includes compressing two opposing surfaces that extend along a length of said pump assembly.

25. A method as set forth in claim 22, wherein compressing includes compressing two opposing surfaces that extend along a width of said pump assembly.

26. A method as set forth in claim 20, wherein said pump assembly has a deflation actuator positioned within said pump assembly, said deflation actuator extending along the length of said pump assembly.

27. A method as set forth in claim 26, wherein said deflation actuator includes a valve actuation bar.

28. An inflatable implantable prosthesis comprising:
a pump assembly;
said pump assembly including a pump bulb;
said pump assembly including at least one internal check valve in a pathway extending from said pump bulb to an inflatable portion of said prosthetic;
said pump assembly including an actuator arm mechanically linking any randomly selected external surface of said pump assembly to one end of said at least one internal check valve.

29. A prosthesis as set forth in claim 28, wherein said actuator arm includes a first portion that extends along a length of said pump assembly and a second portion that extends at an angle to said first portion toward said at least one internal check valve.

30. A prosthesis as set forth in claim 28, wherein a portion of said pump assembly has an external textured surface different than an external surface of the pump bulb.

31. A prosthesis as set forth in claim 28, wherein said pump bulb is of a different size and shape from the rest of the pump assembly.

32. A method of making a pump and valve assembly for an inflatable prosthesis, comprising:
providing a valve block having at least one actuable valve;
providing a shell including a pump bulb component; and
attaching the shell to the valve block to complete the pump and valve assembly.

33. A method of manufacturing a pump and valve assembly for an inflatable prosthesis, comprising:
molding a unitary valve block;
inserting at least one valve;
molding a unitary shell including a pump bulb component; and
joining the shell to the valve block to complete the pump and valve assembly without requiring any other components to be joined thereto.

34. A pump and valve assembly for an inflatable prosthesis, comprising:
a unitary molded valve block; and
a unitary molded shell attached to the valve block wherein the shell include a pump bulb.
